(19) United States
(12) Patent Application Publication
(10) Pub. No.: US 2016/0165878 A1
(43) Pub. Date: Jun. 16, 2016

(54) SEMINAL PLASMA COMPLEX QUALITY CONTROL MATERIAL, PREPARATION METHOD AND KIT THEREOF

(71) Applicant: BRED LIFE SCIENCE TECHNOLOGY INC, Guangdong (CN)

(72) Inventors: Hao CHENG, Shenzhen, Guangdong Province (CN); Jiachun HU, Shenzhen, Guangdong Province (CN); Sien TAO, Shenzhen, Guangdong Province (CN); Hongfang YANG, Shenzhen, Guangdong Province (CN); Shaojun DENG, Shenzhen, Guangdong Province (CN)

(73) Assignee: BRED LIFE SCIENCE TECHNOLOGY INC, Shenzhen, Guangdong Province (CN)

(21) Appl. No.: 14/910,217

(22) PCT Filed: Mar. 13, 2015
(86) PCT No.: PCT/CN2015/074248

§ 371 Date: Feb. 4, 2016

(30) Foreign Application Priority Data
Apr. 11, 2014 (CN) 201410145716.0

(51) Int. Cl. A01N 1/02; C12N 9/96

(52) U.S. Cl. CPC A01N 1/021; C12N 9/96

(57) ABSTRACT

The present invention provides a seminal plasma complex quality control serum, which is freeze-dried powders prepared by mixing a quality control serum preservation solution and a seminal plasma stock solution; wherein the quality control serum preservation solution is an aqueous solution containing 0.47% to 0.67% saccharides, 0.07% to 0.17% salts and 0.63% to 0.83% enzyme stabilizers; the seminal plasma stock solution is the seminal plasma obtained after the direct centrifugation of completely liquefied fresh human semen; and a mixing ratio by volume of the quality control serum preservation solution to the seminal plasma stock solution is 1:100 to 100:1. According to the present invention, a specific quality control serum preservation solution is used, then mixed with the seminal plasma at a suitable ratio by volume and finally subjected to freeze-drying treatment.

### TECHNICAL FIELD

The present invention relates to in vitro detection reagents, and more particularly relates to a seminal plasma complex quality control serum, and a preparation method and a reagent kit thereof.

### BACKGROUND

Detection of biochemical indicators of semen lacks an experimental quality control serum, such that standard work of the biochemical analysis of semen cannot be advanced. A large number of substances in semen are very unstable in vitro and hard to be preserved, which even increases the technical difficulty in the preparation of a seminal plasma quality control serum.

In the related art, the quality control serums used by reagent kits for detection of various detection items of the seminal plasma are only dedicated quality control serums directed to their respective detection items, but are not prepared from the seminal plasma. At present, no quality control serum prepared from the seminal plasma has been used in the reagent kits for detection of various detection items of the seminal plasma.

### SUMMARY

The technical problem to be solved by the present invention is to provide a seminal plasma complex quality control serum for biochemical immunoassay and experimental quality control of the seminal plasma, which uses seminal plasma as a matrix and features stable properties and simple use. The present invention further provides a method for the preparation of the seminal plasma complex quality control serum and a method for quality control of biochemical detection items of seminal plasma by using the seminal plasma detection reagent kit containing the seminal plasma complex quality control serum.

The present invention provides a seminal plasma complex quality control serum, which is freeze-dried powders prepared by mixing a quality control serum preservation solution and a seminal plasma stock solution; wherein the quality control serum preservation solution is an aqueous solution containing 0.47% to 0.67% saccharides, 0.07% to 0.17% salts and 0.63% to 0.83% enzyme stabilizers; the seminal plasma stock solution is the seminal plasma obtained after the direct centrifugation of completely liquefied fresh human semen; and a mixing ratio by volume of the quality control serum preservation solution to the seminal plasma stock solution is 1:100 to 100:1.

The saccharides are sucrose, the salts are sodium chloride, and the enzyme stabilizers are glycine.

The quality control serum preservation solution is an aqueous solution containing 0.57% sucrose, 0.12% sodium chloride and 0.73% glycine; and the ratio by volume of the quality control serum preservation solution to the seminal plasma stock solution is 1:1.

The present invention further provides a method for the preparation of the seminal plasma complex quality control serum, which comprises the following steps: preparation of a quality control serum preservation solution: weighing saccharides, salts and enzyme stabilizers, and adding purified water until the saccharides, salts and enzyme stabilizers are fully dissolved to obtain the quality control serum preservation solution having the above-defined component percentages; acquisition of a seminal plasma stock solution: taking several parts of completely liquefied fresh human semen with HCV, which has negative results in HIV1+2, HBSAg and syphilis detection; obtaining the seminal plasma stock solution via centrifugation, and uniformly mixing the obtained seminal plasma stock solution; and acquisition of a seminal plasma complex quality control serum via mixing and freeze-drying: uniformly mixing the seminal plasma stock solution with the quality control serum preservation solution according to the above-defined ratio by volume, and then preparing the mixed solution into freeze-dried powders in a vacuum freeze-dryer, that is, the seminal plasma complex quality control serum.

The freeze-drying in the vacuum freeze-dryer comprises the following steps: starting the vacuum freeze-dryer, setting a freeze-drying curve to −40° C. and maintaining this temperature for 2 hours, pumping for 2 hours and starting vacuum to adjust the temperature to −40° C. and maintaining this temperature for 16 hours, raising the temperature to 0° C. and maintaining this temperature for 3 hours, raising the temperature to 30° C. and maintaining this temperature for 5 hours, and finally exhausting gas and sealing the cover, to obtain a powder-like seminal plasma complex quality control serum.

The present invention further provides a seminal plasma detection reagent kit containing the seminal plasma complex quality control serum as defined above, comprising: a seminal plasma complex quality control serum; a reagent for detection of biochemical indicators of the seminal plasma; and a reagent kit instruction specifying fixed value ranges of the biochemical indicators of the seminal plasma; wherein the fixed value ranges of the biochemical indicators of the seminal plasma are obtained by the following method: uniformly mixing the seminal plasma stock solution and the quality control serum preservation solution, charging the mixed solution into two 2 ml flasks, preparing the mixed solution into a powder-like seminal plasma complex quality control serum in a vacuum freeze-dryer, taking 24 flasks of the seminal plasma complex quality control serums, adding 2 ml of purified water into each flask for full re-dissolution, performing detection three times, each time eight flasks by using reagents for detection of the biochemical indicators of the seminal plasma, obtaining via statistical analysis an average value X and a standard difference S of each biochemical indicator of the seminal plasma, and obtaining a fixed value range X±2S of the biochemical indicator in the reagent kit by adding the average value to or subtracting the average value from a value twice of the standard difference.

The reagent for detection of the biochemical indicators of the seminal plasma is one or a plurality of: a reagent kit for quantitative determination of elastase of seminal plasma, a reagent kit for quantitative determination of zinc of seminal plasma, a reagent kit for quantitative determination of lemon acid of seminal plasma, a reagent kit for quantitative determination of neutral α-glucosidase of seminal plasma, a reagent kit for quantitative determination of lactate dehydrogenase X isoenzyme of seminal plasma, a reagent kit for quantitative determination of acid phosphatase of seminal plasma, a reagent kit for quantitative determination of fructose of seminal plasma, and a reagent kit for quantitative determination of acrosomal enzyme of seminal plasma.

A method for quality control of biochemical detection items of seminal plasma by using the seminal plasma detection reagent kit containing the seminal plasma complex quality control serum as defined above is provided, comprising the following steps:

Step 1: Dissolution of a Seminal Plasma Complex Quality Control Serum

taking 1 flask of the seminal plasma complex quality control serum, dissolving the seminal plasma complex quality control serum with 2 ml of purified water or a physiological saline solution or another dissolving solution, placing still the serum or incubating the serum at 37° C. or incubating the serum in a shaking manner or shaking the serum at a constant temperature to fully dissolve the serum;

Step 2: Acquisition of Detected Value

detecting the seminal plasma complex quality control serum by using the reagent for detection of biochemical indicators of the seminal plasma, to obtain the detected value; and

Step 3: Comparison of the Detected Value with a Fixed Value Range

comparing the detected values with the fixed value ranges of the biochemical indicators of the seminal plasma in the seminal plasma complex quality control serum; wherein if the detected value is within the fixed value range X±2S, the detection result is reliable, and if the detected value exceeds the fixed range value X±2S, the tested value obtained in this detection is not reliable, causes need to be identified and test needs to be re-conducted.

According to the present invention, a specific quality control serum preservation solution is used, then mixed with the seminal plasma at a suitable ratio by volume and finally subjected to freeze-drying treatment. As such, various biochemical indicators of the seminal plasma are more stable, thereby effectively addressing the defect that the substances in the seminal plasma are not stable in vitro and are hard to be preserved. Instead, such prepared seminal plasma complex quality control serum features stable properties and simple use, and may be used for quality control of biochemical detection items of seminal plasma.

### DETAILED DESCRIPTION

The present invention provides a seminal plasma complex quality control serum, which comprises a quality control serum preservation solution and a seminal plasma stock solution; wherein the quality control serum preservation solution is an aqueous solution containing 0.47% to 0.67% saccharides, 0.07% to 0.17% salts and 0.63% to 0.83% enzyme stabilizers (wherein the percentage is m/v, and the unit is g/ml).

A mixing ratio by volume of the quality control serum preservation solution to the seminal plasma stock solution is 1:100 to 100:1.

The seminal plasma stock solution herein is the seminal plasma obtained after direct centrifugation of completely liquefied fresh human semen.

The saccharides may be sucrose, fructose, maltose, or glucose; the salts may be sodium chloride, potassium chloride, calcium chloride, sodium carbonate, potassium carbonate, sodium bicarbonate, potassium bicarbonate, disodium hydrogen phosphate, monosodium phosphate, dipotassium hydrogen phosphate, or monopotassium phosphate; and the enzyme stabilizers may be glycine, glycerol, polyvinylamine, mannitol, or toluene.

The method for the preparation of the seminal plasma complex quality control serum comprises:

Preparation of a quality control serum preservation solution: weighing the above specified amounts of saccharides, salts and enzyme stabilizers, and mixing and adding 100 ml purified water until the saccharides, salts and enzyme stabilizers are fully dissolved to obtain the quality control serum preservation solution having the above-defined component percentages.

Acquisition of a seminal plasma stock solution: taking several parts of completely liquefied fresh human semen, and selecting parts of completely liquefied fresh human semen with HCV, HIV1+2, HBSAg and syphilis detections being negative as a starting material, obtaining the seminal plasma stock solution via centrifugation (freeze-preserved semen or seminal plasma stock solution may also be used as the starting material, wherein the HCV, HIV1+2, HBSAg and syphilis detections are also needed), and uniformly mixing the obtained seminal plasma stock solution to obtain the seminal plasma stock solution.

Acquisition of a seminal plasma complex quality control serum via mixing and freeze-drying: taking 100 ml of seminal plasma stock solution, uniformly mixing the 100 ml of seminal plasma stock solution with the quality control serum preservation solution at a mixing ratio by volume of 1:100 to 100:1, charging the mixed solution into two 2 ml flasks, and preparing the mixed solutions into freeze-dried powders in a freeze-dryer; wherein the freeze-drying in the vacuum freeze-dryer comprises the following steps: starting the vacuum freeze-dryer, setting a freeze-drying curve to −40° C. and maintaining this temperature for 2 hours, pumping for 2 hours and starting vacuum to adjust the temperature to −40° C. and maintaining this temperature for 16 hours, raising the temperature to 0° C. and maintaining this temperature for 3 hours, raising the temperature to 30° C. and maintaining this temperature for 5 hours, and finally exhausting gas and sealing the cover, to obtain a powder-like seminal plasma complex quality control serum.

A fixed value range is defined for the biochemical indicators of the seminal plasma by using the above seminal plasma complex quality control serum. The biochemical indicators of the seminal plasma may be preferably one or a plurality of fructose of seminal plasma, neutral α-glucosidase of seminal plasma, zinc of seminal plasma, acid phosphatase of seminal plasma, lemon acid of seminal plasma, elastase of seminal plasma, lactate dehydrogenase X isoenzyme of seminal plasma, and acrosomal enzyme of seminal plasma.

The present invention further provides a method for quality control of biochemical detection items of seminal plasma by using the seminal plasma detection reagent kit containing the seminal plasma complex quality control serum as defined above, comprising the following steps:

Step 1: Dissolution of a Seminal Plasma Complex Quality Control Serum

dissolving the seminal plasma complex quality control serum with purified water or a physiological saline solution or another dissolving solution, placing still the serum or incubating the serum at 37° C. or incubating the serum in a shaking manner or shaking the serum at a constant temperature to fully dissolve the serum;

Step 2: Acquisition of Detected Value

detecting the seminal plasma complex quality control serum by using the method for detecting a common seminal plasma sample using the reagent for detection of biochemical indicators, to obtain the detected value; and

Step 3: Comparison of the Detected Value with a Fixed Value Range

comparing the detected values with the fixed value ranges of the seminal plasma complex quality control serum; wherein if the detected value is within the fixed value range, the detection result is reliable, and if the detected value exceeds the fixed value range, the tested value obtained in this detection is not reliable, causes need to be identified and test needs to be re-conducted.

In addition, the values obtained via several detections may be statistically collected. If several values continuously deviate from the fixed values, even though these values are within the fixed value range, the detection result is not reliable. In this case, systematic deviations are present.

### Embodiment 1

This embodiment provides a seminal plasma complex quality control serum, comprising a quality control serum preservation solution and a seminal plasma stock solution; wherein the quality control serum preservation solution is an aqueous solution containing sucrose, sodium chloride and glycine; and the quality control serum preservation solution and the seminal plasma stock solution are mixed at a ratio by volume of 1:1, to form dry powders via freeze-drying.

This embodiment further provides a method for the preparation of the seminal plasma complex quality control serum as defined above, comprising the following steps:

Step 1: Preparation of a Quality Control Serum Preservation Solution

weighing 0.7032 g of sucrose, 0.1205 g of sodium chloride and 0.5713 g of glycine, and adding purified water to 100 ml for full dissolution, to obtain the quality control serum preservation solution;

Step 2: Acquisition of a Seminal Plasma Stock Solution

taking several parts of completely liquefied fresh human semen, and selecting parts of completely liquefied fresh human semen with HCV, HIV1+2, HBSAg and syphilis detections being negative as a starting material, obtaining the seminal plasma stock solution via centrifugation for 20 minutes under 3000 g (freeze-preserved semen or semen plasma stock solution may also be used as the starting material, wherein the HCV, HIV1+2, HBSAg and syphilis detections are also needed), and uniformly mixing the obtained seminal plasma stock solution to obtain the seminal plasma stock solution; and

Step 3: Acquisition of a Seminal Plasma Complex Quality Control Serum Via Mixing and Freeze-Drying

taking 100 ml of seminal plasma stock solution, uniformly mixing the 100 ml of seminal plasma stock solution with the quality control serum preservation solution at a mixing ratio by volume of 1:1, charging the mixed solution into two 2 ml flasks, and preparing the mixed solutions into freeze-dried powders in a freeze-dryer; wherein the freeze-drying in the vacuum freeze-dryer comprises the following steps: starting the vacuum freeze-dryer, setting a freeze-drying curve to −40° C. and maintaining this temperature for 2 hours, pumping for 2 hours and starting vacuum to adjust the temperature to −40° C. and maintaining this temperature for 16 hours, raising the temperature to 0° C. and maintaining this temperature for 3 hours, raising the temperature to 30° C. and maintaining this temperature for 5 hours, and finally exhausting gas and sealing the cover, to obtain a powder-like seminal plasma complex quality control serum.

### Embodiment 2

This embodiment differs from Embodiment 1 in that:

The quality control serum preservation solution an aqueous solution containing polyvinylamine, sodium carbonate and glucose; and the quality control serum preservation solution and the seminal plasma stock solution are mixed at a ratio by volume of 1:80, to form dry powders via freeze-drying.

The method for the preparation of the quality control serum preservation solution is: weighing 0.8134 g of polyvinylamine, 0.0954 g of sodium carbonate and 0.6543 g of glucose, and adding purified water to 100 ml for full dissolution, to obtain the quality control serum preservation solution.

### Embodiment 3

This embodiment differs from Embodiment 1 in that:

The quality control serum preservation solution an aqueous solution containing toluene, potassium bicarbonate and maltose; and the quality control serum preservation solution and the seminal plasma stock solution are mixed at a ratio by volume of 60:1, to form dry powders via freeze-drying.

The method for the preparation of the quality control serum preservation solution is: weighing 0.6835 g of toluene, 0.0812 g of potassium bicarbonate and 0.4832 g of maltose, and adding purified water to 100 ml for full dissolution, to obtain the quality control serum preservation solution.

### Embodiment 4

This embodiment provides a seminal plasma detection reagent kit containing the seminal plasma complex quality control serum as defined in claim 1, 2 or 3, comprising:

a seminal plasma complex quality control serum;

a reagent for detection of biochemical indicators of the seminal plasma (which is one or a plurality of: a reagent kit for quantitative determination of elastase of seminal plasma, a reagent kit for quantitative determination of zinc of seminal plasma, a reagent kit for quantitative determination of lemon acid of seminal plasma, a reagent kit for quantitative determination of neutral α-glucosidase of seminal plasma, a reagent kit for quantitative determination of lactate dehydrogenase X isoenzyme of seminal plasma, a reagent kit for quantitative determination of acid phosphatase of seminal plasma, a reagent kit for quantitative determination of fructose of seminal plasma, and a reagent kit for quantitative determination of acrosomal enzyme of seminal plasma); and

a reagent kit instruction specifying fixed value ranges of the biochemical indicators of the seminal plasma in the above eight reagent kits.

The fixed value ranges of the biochemical indicators of the seminal plasma in the above eight reagent kits are obtained by the following method (using Embodiment 1 as an example):

taking 24 flasks of the seminal plasma complex quality control serum (freeze-dried powders) prepared in Embodiment 1, adding 2 ml of purified water into each flask for full re-dissolution, and performing detection three times, each time eight flasks; wherein the detection items of each flask of freeze-dried powders of seminal plasma complex quality control serum and the corresponding detection reagents are as listed in Table 1.

TABLE 1

| Detection item | Detection reagent | Detection method | Manufacturer | Model |
|---|---|---|---|---|
| Elastase | Reagent kit for | Follow the | Shenzhen | 96 |
| | quantitative | reagent kit | BRED Life | tests/kit |
| | determination of | instruction | Science | |
| | elastase of seminal | | Technology | |
| | plasma (enzyme | | Inc. | |
| | linked | | | |
| | immunosorbent | | | |
| | assay) | | | |
| Zinc | Reagent kit for | Follow the | Shenzhen | 40 |
| | quantitative | reagent kit | BRED Life | tests/kit |
| | determination of zinc | instruction | Science | |
| | of seminal plasma | | Technology | |
| | (5-Br-PAPS | | Inc. | |
| | coloration method) | | | |
| Lemon acid | Reagent kit for | Follow the | Shenzhen | 40 |
| | quantitative | reagent kit | BRED Life | tests/kit |
| | determination of | instruction | Science | |
| | lemon acid of | | Technology | |
| | seminal plasma | | Inc. | |
| | (enzyme method) | | | |
| Neutral | Reagent kit for | Follow the | Shenzhen | 40 |
| α-glucosidase | quantitative | reagent kit | BRED Life | tests/kit |
| | determination of | instruction | Science | |
| | neutral | | Technology | |
| | α-glucosidase of | | Inc. | |
| | seminal plasma | | | |
| | (improved Cooper | | | |
| | method) | | | |
| Lactate | Reagent kit for | Follow the | Shenzhen | 40 |
| dehydrogenase | quantitative | reagent kit | BRED Life | tests/kit |
| X isoenzyme | determination of | instruction | Science | |
| | lactate | | Technology | |
| | dehydrogenase X | | Inc. | |
| | isoenzyme of | | | |
| | seminal plasma | | | |
| | (velocity method) | | | |
| Acid | Reagent kit for | Follow the | Shenzhen | 40 |
| phosphatase | quantitative | reagent kit | BRED Life | tests/kit |
| | determination of | instruction | Science | |
| | acid phosphatase of | | Technology | |
| | seminal plasma | | Inc. | |
| | (nitrophenyl | | | |
| | phosphoric acid | | | |
| | method) | | | |
| Fructose | Reagent kit for | Follow the | Shenzhen | 40 |
| | quantitative | reagent kit | BRED Life | tests/kit |
| | determination of | instruction | Science | |
| | fructose of seminal | | Technology | |
| | plasma (enzyme | | Inc. | |
| | method) | | | |
| Acrosomal | Reagent kit for | Follow the | Shenzhen | 30 |
| enzyme | quantitative | reagent kit | BRED Life | tests/kit |
| | determination of | instruction | Science | |
| | acrosomal enzyme of | | Technology | |
| | seminal plasma | | Inc. | |
| | (solid phase BAPNA | | | |
| | method) | | | |

Data is statistically collected to analyze an average value and a standard difference of each detection item. The average value is a fixed value (X) of the item in the reagent kit, and a fixed value range (X±2S) of the item in the reagent kit is obtained by adding the average value to or subtracting the average value from a value twice of the standard difference. The experimental results are as listed in Table 2.

TABLE 2

| | | Fixed | Standard | | |
|---|---|---|---|---|---|
| | Detection | value | difference | Lower limit | Upper limit |
| Detection item | method | (X) | (S) | (X − 2S) | (X + 2S) |
| Lactate | Velocity | 753.64 | 43.79 | 666.06 | 841.22 |
| dehydrogenase X | method | | | | |
| isoenzyme (U/L) | | | | | |
| Acid phosphatase | Nitrophenyl | 540.11 | 45.6 | 448.91 | 631.31 |
| (U/ml) | phosphoric | | | | |
| | acid method | | | | |
| Neutral | Improved | 9.1917 | 0.675 | 7.8417 | 10.5417 |
| α-glucosidase | Cooper | | | | |
| (U/L) | method | | | | |
| Lemon acid | Enzyme | 11.5674 | 0.5996 | 10.3682 | 12.7666 |
| (mmol/L) | method | | | | |
| Zinc (mmol/L) | 5-Br-PAPS | 1.1155 | 0.0887 | 0.9381 | 1.2929 |
| | method | | | | |
| Acrosomal | Improved | 141.57 | 10.49 | 120.59 | 162.55 |
| enzyme (μIU/5 μl) | BAPNA | | | | |
| | method | | | | |
| Elastase (ng/ml) | ELISA | 673.71 | 69.81 | 534.09 | 813.33 |
| | sandwich | | | | |
| | method | | | | |
| Fructose | Enzyme | 7.7327 | 0.5816 | 6.5695 | 8.8959 |
| (mmol/L)* | method | | | | |

### Embodiment 5

This embodiment provides a method for quality control of biochemical detection items of seminal plasma by using the seminal plasma detection reagent kit containing the seminal plasma complex quality control serum as defined in Embodiment 4, comprising the following steps:

Step 1: Dissolution of a Seminal Plasma Complex Quality Control Serum

taking 1 flask of the seminal plasma complex quality control serum, dissolving the seminal plasma complex quality control serum with 2 ml of purified water or a physiological saline solution or another dissolving solution, placing still the serum or incubating the serum at 37° C. or incubating the serum in a shaking manner or shaking the serum at a constant temperature to fully dissolve the serum;

Step 2: Acquisition of Detected Value

detecting the seminal plasma complex quality control serum by using the method for detecting a common seminal plasma sample using the eight reagent kits for detection of biochemical indicators, to obtain the detected value;

Step 3: Comparison of the Detected Value with a Fixed Value Range

comparing the detected values with the fixed value ranges of the biochemical indicators of the seminal plasma in the seminal plasma complex quality control serum; wherein if the detected value is within the fixed value range X±2S, the detection result is reliable, and if the detected value exceeds the fixed range value X±2S, the tested value obtained in this detection is not reliable, causes need to be identified and test needs to be re-conducted.

### Embodiment 6

In this embodiment, property evaluation and detection are conducted in the seminal plasma complex quality control serum.

1. Appearance Detection

Three batches of seminal plasma quality control serums are obtained by using the preparation method in Embodiment 1, and 24 flasks of freeze-dried powders of the seminal plasma complex quality control serums are taken. The result shows that the three batches of freeze-dried powers are all light yellow and loose powders via naked eye observation. 2 ml of purified water is added into each flask for re-dissolution to obtain a clarified liquid, without any clot, which complies with the requirements of the reagent kits.

2. Accuracy Detection of the Assigned Value

Three batches of seminal plasma complex quality control serums are obtained by using the preparation method in Embodiment 1, and one flask of seminal plasma complex quality control serum is randomly taken from each batch. Detection is carried out using the detection items and corresponding detection reagents listed in Table 1. The detection results are as listed in Table 3.

TABLE 3

| | | Detection | |
|---|---|---|---|
| Batch | Detection item | result | Fixed value range |
| 1 | Zinc (mmol/L) | 1.30 | 1.123-1.554 |
| | Acid phosphatase (U/ml) | 536.47 | 406.161-583.731 |
| | Elastase (ng/ml) | 554.60 | 485.669-740.565 |
| | Acrosomal enzyme | 137.70 | 129.447-174.536 |
| | (μIU/5 μl) | | |
| | Fructose (mmol/L) | 6.35 | 5.647-7.43 |
| | Lemon acid (mmol/L) | 8.39 | 8.173-10.313 |
| | Neutral α-glucosidase (U/L) | 7.8 | 6.408-8.825 |
| | Lactate dehydrogenase X | 824.33 | 736.372-936.045 |
| | isoenzyme (U/L) | | |
| 2 | Zinc (mmol/L) | 0.77 | 0.702-0.963 |
| | Acid phosphatase (U/ml) | 647.34 | 521.252-739.248 |
| | Elastase (ng/ml) | 681.35 | 602.08-914.403 |
| | Acrosomal enzyme | 229.40 | 186.486-249.614 |
| | (μIU/5 μl) | | |
| | Fructose (mmol/L) | 5.45 | 5.018-6.712 |
| | Lemon acid (mmol/L) | 8.67 | 7.536-9.549 |
| | Neutral α-glucosidase (U/L) | 13.46 | 11.467-15.591 |
| | Lactate dehydrogenase X | 736.63 | 623.241-805.009 |
| | isoenzyme (U/L) | | |
| 3 | Zinc (mmol/L) | 0.97 | 0.865-1.206 |
| | Acid phosphatase (U/ml) | 387.99 | 322.089-472.911 |
| | Elastase (ng/ml) | 840.15 | 754.368~1111.24 |
| | Acrosomal enzyme | 134.99 | 104.589-143.094 |
| | (μIU/5 μl) | | |
| | Fructose (mmol/L) | 8.30 | 7.469-9.59 |
| | Lemon acid (mmol/L) | 13.15 | 12.645-15.56 |
| | Neutral α-glucosidase (U/L) | 8.92 | 8.372-10.994 |
| | Lactate dehydrogenase X | 1079.07 | 935.219-1170.89 |
| | isoenzyme (U/L) | | |

As seen from the detection experimental result of value assignment analysis, casual inspection results of the three batches all comply with the value range of the reagent kits, and comply with the requirements of the reagent kits.

3. Homogeneity Detection in the Flask

Three batches of seminal plasma complex quality control serums are obtained by using the preparation method in Embodiment 1, and one flask of seminal plasma complex quality control serum is randomly taken from each batch. Detection is carried out by using the detection items and corresponding detection reagents listed in Table 1, and the detection is repeated for 10 times to calculate the average value 0, the standard difference (SD) and the coefficient of variation (CV) of the detection results. Detection non-precision in the flask is represented by using the coefficient of variation (CV). The experimental results are as listed in Table 4.

TABLE 4

| Batch | Coefficient of variation (CV) |
|---|---|
| 1 | 7.9% |
| 2 | 7.5% |
| 3 | 7.6% |

As seen from the detection results, the maximum value of the coefficients of variation (CV) of the three batches is 7.9%, which complies with the requirement that the detected coefficient of variation (CV) in the flask of the reagent kits shall be less than or equal to 8%.

4. Non-Precision Detection Between Flasks

Three batches of seminal plasma complex quality control serums are obtained by using the preparation method in Embodiment 1, 10 flasks of freeze-dried powders of seminal plasma complex quality control serum are taken from each batch, and detection is carried out by using the detection items and corresponding detection reagents listed in Table 1. Afterwards, 1 flask of freeze-dried powders of seminal plasma complex quality control serum is randomly taken from each batch, and detection is carried out by using the detection items and corresponding detection reagents listed in Table 1 for 10 times, to calculate CV_{between-flask} (%). Detection non-precision between the flasks is represented by using the coefficient of variation (CV). The results are as listed in Table 5.

TABLE 5

| Batch | Detection result (CV) |
|---|---|
| 1 | 9.3% |
| 2 | 8.1% |
| 3 | 5.9% |

As seen from the detection results, the maximum value of the detection results of the three batches is 9.3%, which complies with the requirement that the non-precision between the flasks of the reagent kits shall be less than or equal to 10%.

1. A seminal plasma complex quality control serum, being freeze-dried powders prepared by mixing a quality control serum preservation solution and a seminal plasma stock solution; wherein
the quality control serum preservation solution is an aqueous solution containing 0.47% to 0.67% saccharides, 0.07% to 0.17% salts and 0.63% to 0.83% enzyme stabilizers;
the seminal plasma stock solution is the seminal plasma obtained after the direct centrifugation of completely liquefied fresh human semen; and
a mixing ratio by volume of the quality control serum preservation solution to the seminal plasma stock solution is 1:100 to 100:1.

2. The seminal plasma complex quality control serum according to claim 1, wherein the saccharides are sucrose, the salts are sodium chloride, and the enzyme stabilizers are glycine.

3. The seminal plasma complex quality control serum according to claim 2, wherein the quality control serum preservation solution is an aqueous solution containing 0.57% sucrose, 0.12% sodium chloride and 0.73% glycine; and the ratio by volume of the quality control serum preservation solution to the seminal plasma stock solution is 1:1.

4. A method for the preparation of the seminal plasma complex quality control serum as defined in claim 1, comprising the following steps:
preparation of a quality control serum preservation solution: weighing saccharides, salts and enzyme stabilizers, and adding purified water until the saccharides, salts and enzyme stabilizers are fully dissolved to obtain the quality control serum preservation solution having the above-defined component percentages;
acquisition of a seminal plasma stock solution: taking several parts of completely liquefied fresh human semen with HCV, HIV1+2, HBSAg and syphilis detections being negative, obtaining the seminal plasma stock solution via centrifugation, and uniformly mixing the obtained seminal plasma stock solution; and
acquisition of a seminal plasma complex quality control serum via mixing and freeze-drying: uniformly mixing the seminal plasma stock solution with the quality control serum preservation solution according to the above-defined ratio by volume, and then preparing the mixed solution into freeze-dried powders in a vacuum freeze-dryer, that is, the seminal plasma complex quality control serum.

5. The method for the preparation of the seminal plasma complex quality control serum according to claim 4, wherein the freeze-drying in the vacuum freeze-dryer comprises the following steps: starting the vacuum freeze-dryer, setting a freeze-drying curve to −40° C. and maintaining this temperature for 2 hours, pumping for 2 hours and starting vacuum to adjust the temperature to −40° C. and maintaining this temperature for 16 hours, raising the temperature to 0° C. and maintaining this temperature for 3 hours, raising the temperature to 30° C. and maintaining this temperature for 5 hours, and finally exhausting gas and sealing the cover, to obtain a powder-like seminal plasma complex quality control serum.

6. A seminal plasma detection reagent kit containing the seminal plasma complex quality control serum as defined in claim 1, comprising:
a seminal plasma complex quality control serum;
a reagent for detection of biochemical indicators of the seminal plasma; and
a reagent kit instruction specifying fixed value ranges of the biochemical indicators of the seminal plasma;
wherein the fixed value ranges of the biochemical indicators of the seminal plasma are obtained by the following method:
uniformly mixing the seminal plasma stock solution and the quality control serum preservation solution, charging the mixed solution into two 2 ml flasks, preparing the mixed solution into a powder-like seminal plasma complex quality control serum in a vacuum freeze-dryer, taking 24 flasks of the seminal plasma complex quality control serums, adding 2 ml of purified water into each flask for full re-dissolution, performing detection three times, each time eight flasks by using reagents for detection of the biochemical indicators of the seminal plasma, obtaining via statistical analysis an average value X and a standard difference S of each biochemical indicator of the seminal plasma, and obtaining a fixed value range X±2S of the biochemical indicator in the reagent kit by adding the average value to or subtracting the average value from a value twice of the standard difference.

7. The seminal plasma detection reagent kit containing the seminal plasma complex quality control serum according to claim 6, wherein the reagent for detection of the biochemical indicators of the seminal plasma is one or a plurality of: a reagent kit for quantitative determination of elastase of seminal plasma, a reagent kit for quantitative determination of zinc of seminal plasma, a reagent kit for quantitative determination of lemon acid of seminal plasma, a reagent kit for quantitative determination of neutral α-glucosidase of seminal plasma, a reagent kit for quantitative determination of lactate dehydrogenase X isoenzyme of seminal plasma, a reagent kit for quantitative determination of acid phosphatase of seminal plasma, a reagent kit for quantitative determination of fructose of seminal plasma, and a reagent kit for quantitative determination of acrosomal enzyme of seminal plasma.

8. (canceled)
